(19) United States
(12) Patent Application Publication
(10) Pub. No.: US 2018/0201968 A1
(43) Pub. Date: Jul. 19, 2018

(54) Azidomethyl Ether Deprotection Method

(71) Applicant: NUCLERA NUCLEICS LTD, Stevenage, Hertfordshire (GB)

(72) Inventors: Michael C. Chen, Cambridge (GB); Radu A. Lazar, Cambridge (GB); Jiahao Huang, Cambridge (GB); Gordon R. McInroy, Cambridge (GB)

(21) Appl. No.: 15/744,749

(22) PCT Filed: Jul. 15, 2016
(86) PCT No.: PCT/GB2016/052155

§ 371 Date: Jan. 12, 2018

(30) Foreign Application Priority Data
Jul. 15, 2015 (GB) 1512372.2

(51) Int. Cl. C12P 19/34; C07B 41/02; C12N 9/12

(52) U.S. Cl. CPC C12P 19/34; C12N 9/1264; C12Y 207/07031; C07B 41/02

(57) ABSTRACT

The invention relates to a method of converting an azidomethyl ether substituent to a free hydroxyl group. The invention also relates to methods of nucleic acid synthesis and sequencing comprising the use of nucleotide triphosphates having a 3′-O-azidomethyl substituent, to kits comprising nucleotide triphosphates having a 3′-O-azidomethyl substituent and photoactivatable transition metal complex and to the use of said kits in methods of nucleic acid synthesis and sequencing.

### FIELD OF THE INVENTION

The invention relates to a method of converting an azidomethyl ether substituent to a free hydroxyl group. The invention also relates to methods of nucleic acid synthesis and sequencing comprising the use of nucleotide triphosphates having a 3′-O-azidomethyl substituent, to kits comprising nucleotide triphosphates having a 3′-O-azidomethyl substituent and photoactivatable transition metal complex and to the use of said kits in methods of nucleic acid synthesis and sequencing.

### BACKGROUND OF THE INVENTION

Nucleic acid synthesis is vital to modern biotechnology. The rapid pace of development in the biotechnology arena has been made possible by the scientific community's ability to artificially synthesize DNA, RNA and proteins.

Artificial DNA synthesis—a £1 billion and growing market—allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. While the benefits of DNA synthesis are numerous, an oft-mentioned problem prevents the further growth of the artificial DNA synthesis industry, and thus the biotechnology field. Despite being a mature technology, it is practically impossible to synthesise a DNA strand greater than 200 nucleotides in length, and most DNA synthesis companies only offer up to 120 nucleotides. In comparison, an average protein-coding gene is of the order of 2000-3000 nucleotides, and an average eukaryotic genome numbers in the billions of nucleotides. Thus, all major gene synthesis companies today rely on variations of a ‘synthesise and stitch’ technique, where overlapping 40-60-mer fragments are synthesised and stitched together by PCR (see Young, L. et al. (2004) Nucleic Acid Res. 32, e59). Current methods offered by the gene synthesis industry generally allow up to 3 kb in length for routine production.

The reason DNA cannot be synthesised beyond 120-200 nucleotides at a time is due to the current methodology for generating DNA, which uses synthetic chemistry (i.e., phosphoramidite technology) to couple a nucleotide one at a time to make DNA. As the efficiency of each nucleotide-coupling step is 95.0-99.0% efficient, it is mathematically impossible to synthesise DNA longer than 200 nucleotides in acceptable yields. The Venter Institute illustrated this laborious process by spending 4 years and 20 million USD to synthesise the relatively small genome of a bacterium (see Gibson, D. G. et al. (2010) Science 329, 52-56).

Known methods of DNA sequencing use template-dependent DNA polymerases to add 3′-reversibly terminated nucleotides to a growing double-stranded substrate (see, Bentley, D. R. et al. (2008) Nature 456, 53-59). In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

There is therefore a need to provide improved methods of nucleic acid synthesis and sequencing that is able to overcome the problems associated with currently available methods.

### SUMMARY OF THE INVENTION

According to a first aspect of the invention, there is provided a method of converting an azidomethyl ether substituent to a free hydroxyl group wherein said method comprises the step of exposing a compound having said azidomethyl ether substituent to a photoactivated transition metal complex.

According to a second aspect of the invention, there is a method of nucleic acid synthesis, which comprises the steps of:
(a) providing an initiator sequence;
(b) adding a nucleotide triphosphate having a 3′-O-azidomethyl substituent to said initiator sequence in the presence of terminal deoxynucleotidyl transferase (TdT) or a functional equivalent or fragment thereof;
(c) removal of TdT;
(d) adding a cleavage composition comprising a photoactivatable transition metal complex and a suitable electron donor;
(e) cleaving the 3′-O-azidomethyl substituent by photoactivating said photoactivatable transition metal complex; and
(f) removing the cleavage composition.

According to a further aspect of the invention, there is provided a kit comprising a nucleotide triphosphate having a 3′-O-azidomethyl substituent and a photoactivatable transition metal complex as defined herein, optionally in combination with one or more components selected from: an initiator sequence, TdT, a suitable electron donor and a microfluidic device or chip; further optionally together with instructions for use of the kit in accordance with any of the methods defined herein.

According to a further aspect of the invention, there is provided the use of a kit in a method of nucleic acid synthesis or sequencing, wherein said kit comprises a photoactivatable transition metal complex as defined herein, optionally in combination with one or more components selected from: an initiator sequence, one or more nucleotide triphosphates having a 3′-O-azidomethyl substituent, TdT, a suitable electron donor and a microfluidic device or chip; further optionally together with instructions for use of the kit in accordance with any of the methods defined herein.

According to a further aspect of the invention, there is provided a method of cleaving an azide-containing linker moiety wherein said method comprises the step of exposing a compound having said azide-containing linker moiety to a photoactivated transition metal complex and suitable electron donor.

### BRIEF DESCRIPTION OF THE FIGURES

FIG. 1: Schematic of enzymatic DNA synthesis platform. Starting from the top of the figure, an immobilised strand of DNA with a deprotected 3′-end is exposed to an extension mixture composed of TdT, a base-specific 3′-blocked nucleotide triphosphate, inorganic pyrophosphatase to reduce the buildup of inorganic pyrophosphate, and appropriate buffers/salts for optimal enzyme activity and stability. The protein adds one protected nucleotide to the immobilised DNA strand (bottom of figure). The extension mixture is then removed with wash mixture and optionally recycled. The immobilised (n+1) DNA strand is then washed with a cleavage mixture to cleave the 3′-protecting group, enabling reaction in the next cycle. In the cleavage mixture, denaturant may be present to disrupt any secondary structures. During this step, the temperature may be raised to assist in cleavage and disruption of secondary structures. The immobilised DNA is treated with wash mixture to remove leftover cleavage mixture. Steps 1-4 may be repeated with an appropriate nucleotide triphosphate until the desired oligonucleotide sequence is achieved.

FIG. 2: Chromatogram to show quantitative cleavage of 3′-O-azidomethyl after 15 minutes of irradiation at 450-500 nm. UV trace (top) and extracted ion count (bottom) of the starting material (3′-O-azidomethyl thymidine).

FIG. 3: Chromatogram to show cleavage of 3′-O-azidomethyl thymidine blocking group with light yields natural thymidine. UV trace (top) and extracted ion count (bottom) for the product (thymidine).

FIG. 4: Simplified schematic representation of a column-based flow instrument used in DNA synthesis. A computer (302) controls two pumps and a solution mixing chamber (311). Pump 1 (304) selectively pumps extension solution (301), wash solution (305) or cleavage solution (310) into the mixing chamber. Pump 2 (306) selectively pumps a single 3′-blocked nucleotide triphosphate (TP) solution containing either 3′-blocked A(adenine)TP (303), T(thymine)TP (307), G(guanine)TP (308), or C(cytosine)TP (309) into the chamber. The computer controlled mixing chamber then passes appropriate solution ratios from pump 1 and pump 2 into a column based DNA synthesis chamber (312). A heating element (313) ensures that the DNA synthesis column remains at the necessary temperature for the synthesis process to take place. Upon exiting the DNA synthesis chamber, the reaction solution either enters a recycling vessel (314) for future use, a waste vessel (316) or moves on to a polymerase chain reaction (PCR) step (315) for amplification of the resultant DNA. PCR completion leads to the final product (317).

FIG. 5: Ruthenium-mediated deprotection of azidomethyl reversible terminator in the context of sequencing-by-synthesis. A duplex of ODN_01 and ODN_02 was formed and either left untreated, or treated with ruthenium solution (1 mM tris(bipyridine) ruthenium(II) chloride, 50 mM sodium ascorbate, 100 mM tris-HCl pH 7.5 and exposure to 455 nm light for 5 minutes). The duplexes were immobilised on a streptavidin-coated, black, 96-well plate. The duplexes were incubated with Therminator (2U), ThermoPol Buffer (1×), and either fluorescently labelled cy3-dUTP or cy5-dCTP (20 uM) for 10 minutes at 72° C. Wells were washed five times with wash buffer (20 mM tris-HCl pH 7.5, 1 mM EDTA, 1 M NaCl, 0.05% tween-20) to remove unincorporated dNTPs. (A) shows a schematic for this process and (B) contains a plot of cy3 and cy5 channel fluorescence intensities for ruthenium treatment, normalised against the untreated sample.

### DETAILED DESCRIPTION OF THE INVENTION

According to a first aspect of the invention, there is provided a method of converting an azidomethyl ether substituent to a free hydroxyl group wherein said method comprises the step of exposing a compound having said azidomethyl ether substituent to a photoactivated transition metal complex.

The use described herein has significant advantages, in particular in respect to nucleic acid synthesis. For example the ability to generate single-stranded oligonucleotides in a sequence-controlled, sequential manner.

References to the term ‘azidomethyl ether’ as used herein refer to an azido (N₃) group coupled to a methylene (CH₂) group and an ether (—O—) group. Thus, azidomethyl ether may be chemically written as —O—CH₂—N₃.

References to the term ‘free hydroxyl group’ as used herein refer to a free —OH group.

References to the term ‘photoactivated transition metal complex’ as used herein refers to a transition metal complex that has increased reductive and oxidative power upon photoexcitation.

References to the term ‘transition metal complex’ as used herein refer to a transition metal in combination with a ligand. In one embodiment the transition metal complex comprises a transition metal selected from ruthenium, platinum, palladium, rhodium and osmium. In a further embodiment the transition metal is ruthenium.

In one embodiment the transition metal complex comprises a ligand which is a mono-dentate or bidentate ligand selected from phosphine, thiocyanate, nitrogen, pyridine, phenanthroline, cyclopentadienyl and N-heterocyclic carbine based ligands. In a further embodiment the photoactivated transition metal complex comprises a pyridine ligand, such as a bipyridine ligand. In a further embodiment the transition metal complex is tris(2,2′-bipyridyl)ruthenium(II)).

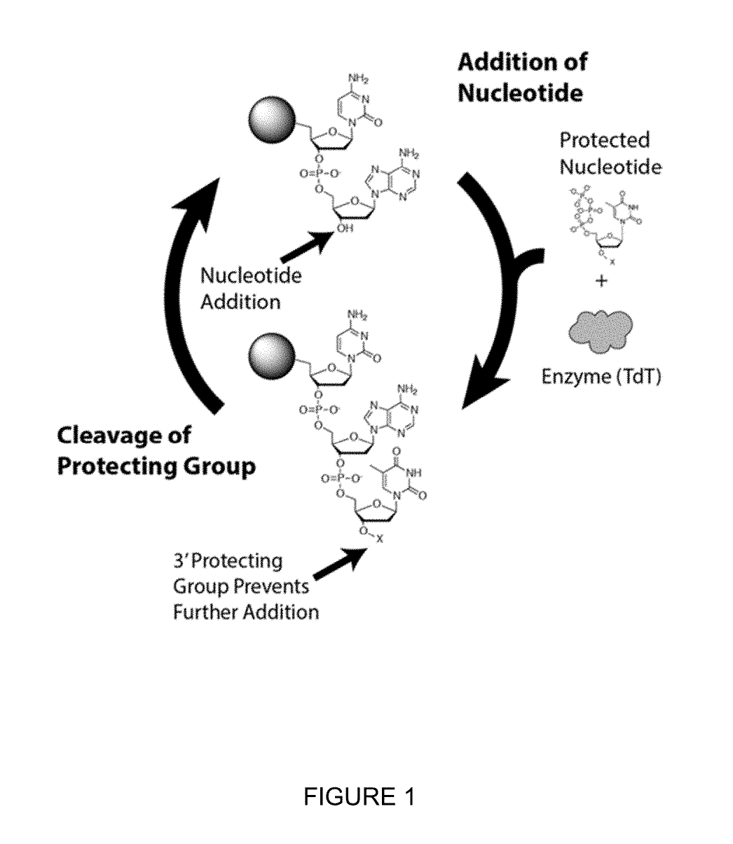

In one embodiment the azidomethyl ether is present on a ribose or deoxyribose sugar moiety. The azidomethyl ether substituent finds particular utility as a protecting group, such as a 2′ or 3′-O-azidomethyl substituent which may be used to reversibly block nucleotide triphosphates in order to control the nucleic acid sequence during TdT-mediated coupling. Once the 2′ or 3′-O-azidomethyl substituent of the nucleotide added to a growing nucleic acid strand is converted to a free hydroxyl group it is readied for subsequent coupling (see FIG. 1). Therefore, in a further embodiment the azidomethyl ether is a 2′ or 3′-O-azidomethyl. This has the advantage of providing a photo-mediated mechanism to control massively parallel oligonucleotide synthesis at a scale that has not previously been achievable.

In one embodiment the method comprises the step of exposing a compound having said azidomethyl ether substituent to a photoactivatable transition metal complex followed by photoactivating said photoactivatable transition metal complex.

References to the term ‘photoactivatable transition metal complex’ as used herein refers to a transition metal complex capable of having increased reductive and oxidative power upon photoexcitation by means of radiant energy and especially light.

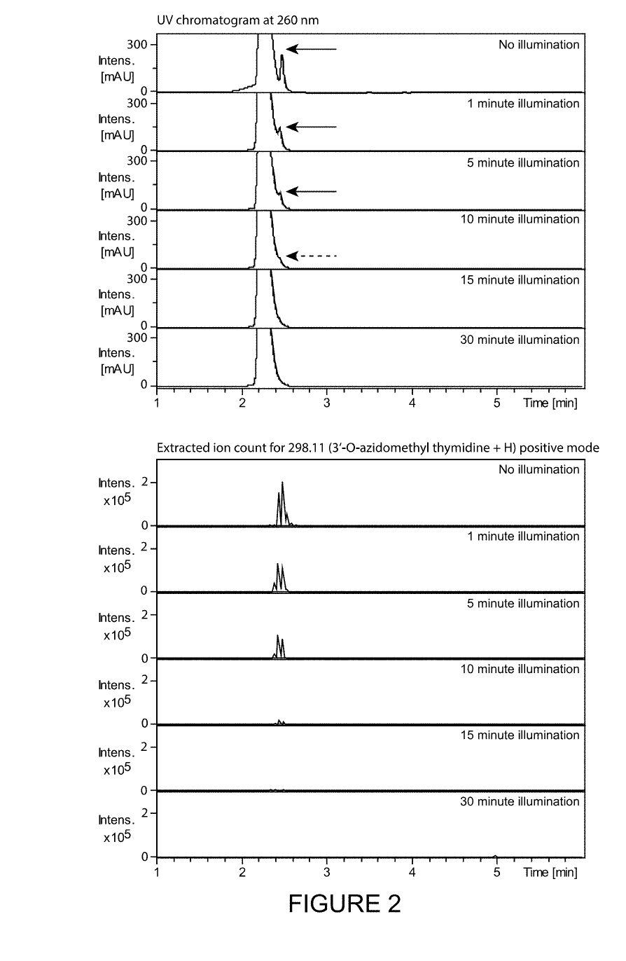

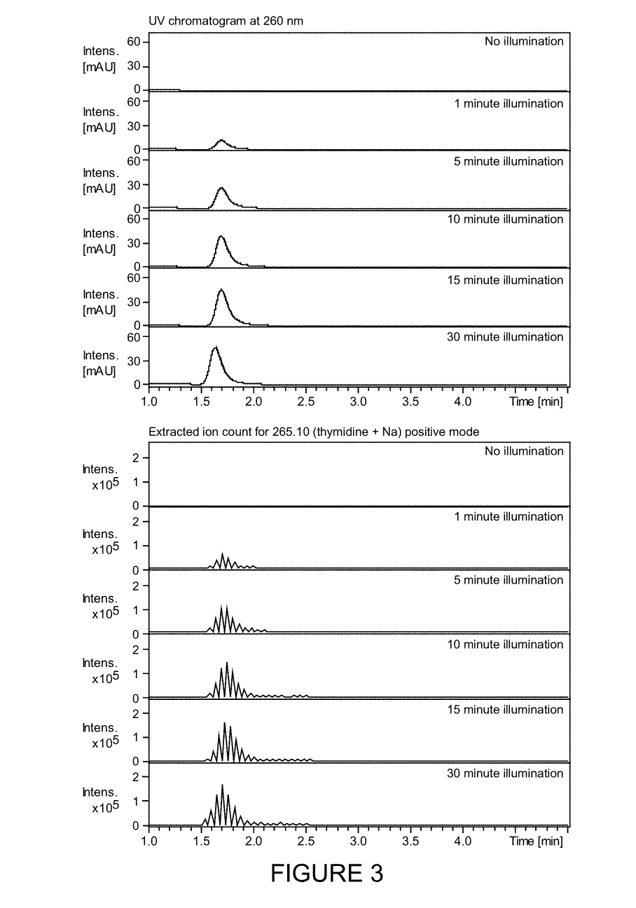

In one embodiment said photoactivating is performed with electromagnetic radiation, such as radiation in the UV or visible range between 400 and 500 nm, such as 450 to 500 nm, in particular 450 nm. Results are presented herein which show successful generation of the free hydroxyl group at these wavelengths (see FIG. 2). In a further embodiment said photoactivating is performed at 452 nm±5 nm. Results are provided herein which demonstrate that this embodiment has the advantage of providing photoactivation without the use of biologically damaging UV-radiation (see FIG. 3).

In one embodiment said photoactivating is performed at a temperature of between 5 and 95° C., such as 20 to 60° C. Results are presented herein which show successful generation of the free hydroxyl group at these temperatures (see FIG. 2). In a further embodiment said photoactivating is performed at 22° C.

In one embodiment said photoactivating is controlled by a digital micromirror device, a photolithographic mask or a laser array. Such applications allow for selective cleaving of a sub-set of a plurality of oligonucleotides with a 2′ or 3′-O-azidomethyl substituent during massively parallel oligonucleotide synthesis on a scale that has not previously been achievable.

References to the term ‘digital micromirror devices’ as used herein refer to devices comprising opto-mechanical and electro-mechanical elements that enable spatial light modulation.

In one embodiment said transition metal complex is used in combination with a suitable electron donor. In a further embodiment the suitable electron donor is sodium ascorbate.

### Nucleic Acid Synthesis Method

In one embodiment of the invention, there is provided a method according to the first aspect of the invention for use in nucleic acid synthesis.

In a further aspect of the invention, there is provided a method of nucleic acid synthesis comprising the steps of:
(a) providing an initiator sequence;
(b) adding a nucleotide triphosphate having a 3′-O-azidomethyl substituent to said initiator sequence in the presence of terminal deoxynucleotidyl transferase (TdT) or a functional equivalent or fragment thereof;
(c) removal of TdT;
(d) adding a cleavage composition comprising a photoactivatable transition metal complex and a suitable electron donor;
(e) cleaving the 3′-O-azidomethyl substituent by photoactivating said photoactivatable transition metal complex; and
(f) removing the cleavage composition.

References herein to a ‘method of nucleic acid synthesis’ include methods of synthesising lengths of DNA (deoxyribonucleic acid) or RNA (ribonucleic acid) wherein a strand of nucleic acid (n) is extended by adding a further nucleotide (n+1). In one embodiment, the nucleic acid is DNA. In an alternative embodiment, the nucleic acid is RNA.

References herein to ‘method of DNA synthesis’ refer to a method of DNA strand synthesis wherein a DNA strand (n) is extended by adding a further nucleotide (n+1). The method described herein provides a novel use of the terminal deoxynucleotidyl transferases of the invention and nucleotide triphosphate having a 3′-O-azidomethyl substituent to sequentially add nucleotides in de novo DNA strand synthesis which has several advantages over the DNA synthesis methods currently known in the art.

An alternative, enzymatic method of nucleic acid synthesis is desirable. Natural enzymes such as DNA polymerases are able to add 50,000 nucleotides before disassociation. However, DNA polymerases require a template strand, thereby defeating the purpose of de novo strand synthesis. Given a free 3′-end and nucleotide triphosphates, recombinant TdTs from Bos taurus and Mus musculus were shown to add ten to several hundred nucleotides onto the 3′-end of a DNA strand. As shown in a paper by Basu, M. et al. (Biochem. Biophys. Res. Commun. (1983) 111, 1105-1112) TdT will uncontrollably add nucleotide triphosphates to the 3′-end of a DNA strand. However, this uncontrolled addition is unsuitable for controlled de novo strand synthesis where a sequence-specific oligonucleotide is required. Thus, commercially available recombinant TdT is used primarily as a tool for molecular biologists to label DNA with useful chemical tags. This enzymatic approach means that the method has the particular advantage of being able to produce DNA strands beyond the 120-200 nucleotide limit of current synthetic DNA synthesis methods. Furthermore, this enzymatic method avoids the need to use strong organic solvents which may be harmful to the environment.

In a further embodiment greater than 1 nucleotide is added by repeating steps (b) to (f).

It will be understood that steps (b) to (f) of the method may be repeated multiple times to produce a DNA or RNA strand of a desired length. Therefore, in one embodiment, greater than 1 nucleotide is added to the initiator sequence, such as greater than 5, 10, 20, 30, 40, 50, 60, 70, 80, 90, 100, 110 or 120 nucleotides are added to the initiator sequence by repeating steps (b) to (e). In a further embodiment, greater than 200 nucleotides are added, such as greater than 300, 400, 500, 600, 700, 800, 900, 1000, 1250, 1500, 1750, 2000, 2250, 2500, 2750, 3000, 4000, 5000, 6000, 7000, 8000, 9000 or 10000 nucleotides.

References herein to ‘nucleotide triphosphates’ refer to a molecule containing a nucleoside (i.e. a base attached to a deoxyribose or ribose sugar molecule) bound to three phosphate groups. Examples of nucleotide triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleotide triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleotide triphosphates, such as artificial nucleosides.

Therefore, references herein to ‘nucleotide triphosphate having a 3′-O-azidomethyl substituent’ refer to nucleotide triphosphates (e.g., dATP, dGTP, dCTP or dTTP) which have an azidomethyl ether substituent at the 3′ position which prevents further addition of nucleotides, i.e., by replacing the 3′-OH group with a 3′-O-azidomethyl group which acts as a protecting group.

References to the term ‘cleavage composition’ as used herein refer to a substance which is able to cleave the azidomethyl substituent from the nucleotide triphosphate to yield a 3′-OH group. In one embodiment said cleavage composition of step (d) comprises a transition metal complex used in combination with a suitable electron donor. In a further embodiment the suitable electron donor is sodium ascorbate.

References to the term ‘initiator sequence’ as used herein refer to a short oligonucleotide with a free 3′-end which the nucleotide triphosphate having a 3′-O-azidomethyl substituent can be attached to. In one embodiment, the initiator sequence is a DNA initiator sequence. In an alternative embodiment, the initiator sequence is an RNA initiator sequence.

References herein to a DNA initiator sequence′ refer to a small sequence of DNA which the 3′ nucleotide triphosphate having a 3′-O-azidomethyl substituent can be attached to, i.e. DNA will be synthesised from the end of the DNA initiator sequence.

In one embodiment, the initiator sequence is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long. In an alternative embodiment, the initiator sequence is between 5 and 20 nucleotides long, such as 5 to 15 nucleotides long, for example 10 to 15 nucleotides long, in particular 12 nucleotides long.

In one embodiment, the initiator sequence is single-stranded. In an alternative embodiment, the initiator sequence is double-stranded. It will be understood by persons skilled in the art that a 3′-overhang (i.e., a free 3′-end) allows for efficient addition.

In one embodiment, the initiator sequence is immobilized on a solid support. This allows TdT and the cleaving agent to be removed (in steps (c) and (f), respectively) without washing away the synthesised nucleic acid. The initiator sequence may be attached to a solid support stable under aqueous conditions so that the method can be easily performed via a flow setup.

In one embodiment, the initiator sequence is immobilized on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In a further embodiment, the initiator sequence is immobilized on a solid support via a chemically-cleavable linker, such as a disulfide, allyl, or azide-masked hemiaminal ether linker. Therefore, in one embodiment, the method additionally comprises extracting the resultant nucleic acid by cleaving the chemical linker through the addition of tris(2-carboxyethyl)phosphine (TCEP) or dithiothreitol (DTT) for a disulfide linker; palladium complexes for an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In an alternative embodiment, the immobilized initiator sequence contains at least one restriction site. Therefore, in a further embodiment, the method additionally comprises extracting the resultant nucleic acid by using a restriction enzyme.

The use of restriction enzymes and restriction sites to cut nucleic acids in a specific location is well known in the art. The choice of restriction site and enzyme can depend on the desired properties, for example whether ‘blunt’ or ‘sticky’ ends are required. Examples of restriction enzymes include: AluI, BamHI, EcoRI, EcoRII, EcoRV, HaeII, HgaI, HindII, HinfI, NotI, PstI, PvuII, SaII, Sau3A, ScaI, SmaI, TaqI and XbaI.

References herein to terminal deoxynucleodidyl transferase (TdT) enzyme include references to purified and recombinant forms of said enzyme.

In one embodiment, the terminal deoxynucleotidyl transferase (TdT) is a natural TdT or non-natural TdT or a functional equivalent or fragment thereof.

It will be understood that the term ‘functional equivalent’ refers to the polypeptides which are different to the exact sequence of the TdTs of the first aspect of the invention, but can perform the same function, i.e., catalyse the addition of a nucleotide triphosphate onto the 3′-end of a DNA strand in a template dependent manner.

In one embodiment, the terminal deoxynucleotidyl transferase (TdT) is a non-natural derivative of TdT, such as a functional fragment or homolog of the TdTs of the first aspect of the invention.

References herein to ‘fragment’ include, for example, functional fragments with a C-terminal truncation, or with an N-terminal truncation. Fragments are suitably greater than 10 amino acids in length, for example greater than 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 amino acids in length.

In one embodiment, the terminal deoxynucleotidyl transferase (TdT) has at least 25% homology with the TdTs of the first aspect of the invention, such as at least 30%, 40%, 50%, 55%, 60%, 65%, 70%, 75%, 80%, 85%, 90%, 95%, 96%, 97%, 98% or 99% homology.

In one embodiment, the terminal deoxynucleotidyl transferase (TdT) is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc., all with appropriate counterions, such as Cl⁻) and inorganic pyrophosphatase (e.g., the Saccharomyces cerevisiae homolog). It will be understood that the choice of buffers and salts depends on the optimal enzyme activity and stability.

The use of an inorganic pyrophosphatase helps to reduce the build-up of pyrophosphate due to nucleotide triphosphate hydrolysis by TdT. Therefore, the use of an inorganic pyrophosphatase has the advantage of reducing the rate of (1) backwards reaction and (2) TdT strand dismutation. Thus, according to a further aspect of the invention, there is provided the use of inorganic pyrophosphatase in a method of nucleic acid synthesis. In one embodiment, the inorganic pyrophosphatase comprises purified, recombinant inorganic pyrophosphatase from Saccharomyces cerevisiae.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, step (e) is performed at a temperature less than 99° C., such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, steps (c) and (f) are performed by applying a wash solution. In one embodiment, the wash solution comprises the same buffers and salts as used in the extension solution described herein. This has the advantage of allowing the wash solution to be collected after step (c) and recycled as extension solution in step (b) when the method steps are repeated.

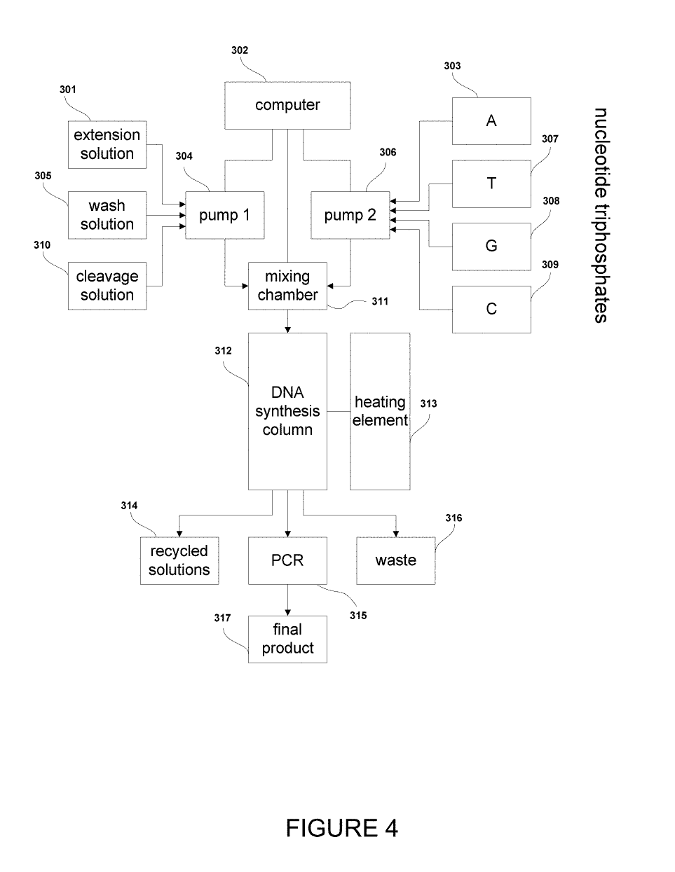

In one embodiment, the method is performed within a flow instrument as shown in FIG. 4, such as a microfluidic or column-based flow instrument. The method described herein can easily be performed in a flow setup which makes the method simple to use. It will be understood that examples of commercially available DNA synthesisers (e.g., MerMade 192E from BioAutomation or H-8 SE from K&A) may be optimized for the required reaction conditions and used to perform the method described herein.

In one embodiment, the method is performed on a plate or microarray setup. This highly parallel process is thus amenable to DNA fragment assembly through standard molecular biology techniques.

In one embodiment, the method additionally comprises amplifying the resultant nucleic acid. Methods of DNA/RNA amplification are well known in the art. For example, in a further embodiment, the amplification is performed by polymerase chain reaction (PCR). This step has the advantage of being able to extract and amplify the resultant nucleic acid all in one step.

The template independent nucleic acid synthesis method described herein has the capability to add a nucleic acid sequence of defined composition and length to an initiator sequence. Therefore, it will be understood by persons skilled in the art, that the method described herein may be used as a novel way to introduce adapter sequences to a nucleic acid library.

If the initiator sequence is not one defined sequence, but instead a library of nucleic acid fragments (for example generated by sonication of genomic DNA, or for example messenger RNA) then this method is capable of de novo synthesis of ‘adapter sequences’ on every fragment. The installation of adapter sequences is an integral part of library preparation for next-generation library nucleic acid sequencing methods, as they contain sequence information allowing hybridisation to a flow cell/solid support and hybridisation of a sequencing primer.

Currently used methods include single stranded ligation, however this technique is limited because ligation efficiency decreases strongly with increasing fragment length. Consequently, current methods are unable to attach sequences longer than 100 nucleotides in length. Therefore, the method described herein allows for library preparation in an alternative fashion to that which is currently possible.

Therefore, in one embodiment, an adapter sequence is added to the initiator sequence. In a further embodiment, the initiator sequence may be a nucleic acid from a library.

In one embodiment, there is provided a method which is performed in a microfluidic device. Thus, according to a further aspect there is provided a method of nucleic acid synthesis in a microfluidic device comprising the steps of:
(a) providing an initiator sequence bound to a surface within a microfluidic device;
(b) adding a nucleotide triphosphate having a 3′-O-azidomethyl substituent to said initiator sequence in the presence of terminal deoxynucleotidyl transferase (TdT) or a functional equivalent or fragment thereof;
(c) removal of TdT;
(d) adding a cleavage composition comprising a photoactivatable transition metal complex and a suitable electron donor;
(e) cleaving the 3′-O-azidomethyl substituent from a sub-set of nucleotide triphosphates having a 3′-O-azidomethyl substituent by selective photoactivation of a sub-set of said photoactivatable transition metal complexes; and
(f) removing the cleavage composition.

References herein to microfluidic device include continuous-flow microfluidic devices, droplet-based microfluidic devices, digital microfluidic devices, microarray devices (such as DNA chips), optofluidic devices and acoustic droplet ejection (ADE) devices.

In a further embodiment, greater than 1 nucleotide is added by repeating steps (b) to (f). In a further embodiment, the surface within the microfluidic device in step (a) may be patterned to yield initiators bound at defined locations. Therefore in a further embodiment the microfluidic device may have a reaction chamber or a plurality of reaction chambers, such as greater than 300, 3000 or 30000 reaction chambers.

In a further embodiment, the selective photoactivation in step (e) is achieved with one or more components selected from: a digital mirror, a photolithographic mask, a light emitting diode (LED), an LED array, a laser, or a laser array. In a yet further embodiment, the selective photoactivation in step (e) is achieved with one or more components selected from: a digital mirror, a photolithographic mask and a laser array.

### Sequencing by Synthesis Method

It will be appreciated that the photochemical methods described herein may be adapted to provide advantageous sequencing methodology, such as sequencing by synthesis methodology. Thus, in one embodiment there is provided a method as defined herein for use in nucleic acid sequencing, such as nucleic acid sequencing by synthesis.

In one embodiment the azidomethyl ether is present on the ribose or deoxyribose sugar moiety of a nucleotide or nucleoside. In a yet further embodiment, the azidomethyl ether is present on the ribose or deoxyribose sugar moiety of a nucleotide or nucleoside and said azidomethyl ether is connected to a detectable tag by an azide-containing linker moiety. Treatment with a reducing agent, such as a photoactivated transition metal complex in the presence of a suitable electron donor, simultaneously exposes a 3′ hydroxyl group and decouples the nucleotide or nucleoside from the detectable tag. This process finds great utility in sequencing by synthesis methods.

Thus, according to a further aspect of the invention, there is provided a sequencing method which comprises cleaving an azide-containing linker moiety wherein said cleaving comprises the step of exposing a compound having said azide-containing linker moiety to a photoactivated transition metal complex.

References to the term ‘azide-containing linker moiety’ as used herein refer to molecules containing the moiety R¹—X—CHN₃—R², wherein X is O, NH, NR³ or S, R¹ is a nucleotide or nucleoside, R² is a detectable label, for example a fluorophore and R³ is an optionally substituted C₁₋₁₀ alkyl group. Under conditions that reduce the azide, this linker decomposes to yield two smaller molecular fragments, thus separating R¹ and R².

### Kits

According to a further aspect of the invention, there is provided a kit comprising a nucleotide triphosphate having a 3′-O-azidomethyl substituent and a photoactivatable transition metal complex as defined herein, optionally in combination with one or more components selected from: an initiator sequence, TdT, a suitable electron donor and a microfluidic device or chip; further optionally together with instructions for use of the kit in accordance with any of the methods defined herein.

According to a further aspect of the invention, there is provided the use of a kit in a method of nucleic acid synthesis, wherein said kit comprises a photoactivatable transition metal complex as defined herein, optionally in combination with one or more components selected from: an initiator sequence, one or more nucleotide triphosphates having a 3′-O-azidomethyl substituent, TdT, a suitable electron donor and a microfluidic device or chip; further optionally together with instructions for use of the kit in accordance with any of the methods defined herein.

According to a further aspect of the invention, there is provided the use of a kit in a method of nucleic acid sequencing, wherein said kit comprises a photoactivatable transition metal complex as defined herein, optionally in combination with one or more components selected from: an initiator sequence, one or more nucleotide triphosphates having a 3′-O-azidomethyl substituent, a suitable electron donor and a microfluidic device or chip; further optionally together with instructions for use of the kit in accordance with any of the methods defined herein.

The following studies and protocols illustrate embodiments of the methods described herein:

### Example 1: A Ruthenium-Mediated Process Unmasks Azidomethyl Moieties to Reveal 3′ Hydroxyls

A solution containing 3′-O-azidomethyl thymidine (100 μM), tris(bipyridine) ruthenium(II)chloride (1 mM), sodium ascorbate (50 mM) and tris-HCl pH 7.4 (100 mM) was irradiated with light (450-500 nm, 1 mW/cm²) for 0, 1, 5, 10, 15 or 30 minutes. Samples were analysed by liquid chromatography mass spectroscopy. FIG. 2 shows the blocking group is efficiently removed under the reaction conditions, and FIG. 3 shows the product following cleavage is indeed natural thymidine with a free 3′-hydroxyl.

Reversible termination of the nascent strand is a key requirement in de novo DNA synthesis methodologies. Ruthenium-mediated removal of the azidomethyl moiety to reveal a 3′ hydroxyl is an example of a suitable reversible termination process.

### Example 2: A Ruthenium-Mediated Process Enables Sequence Identification in a Template Strand for Use in Sequencing-by-Synthesis

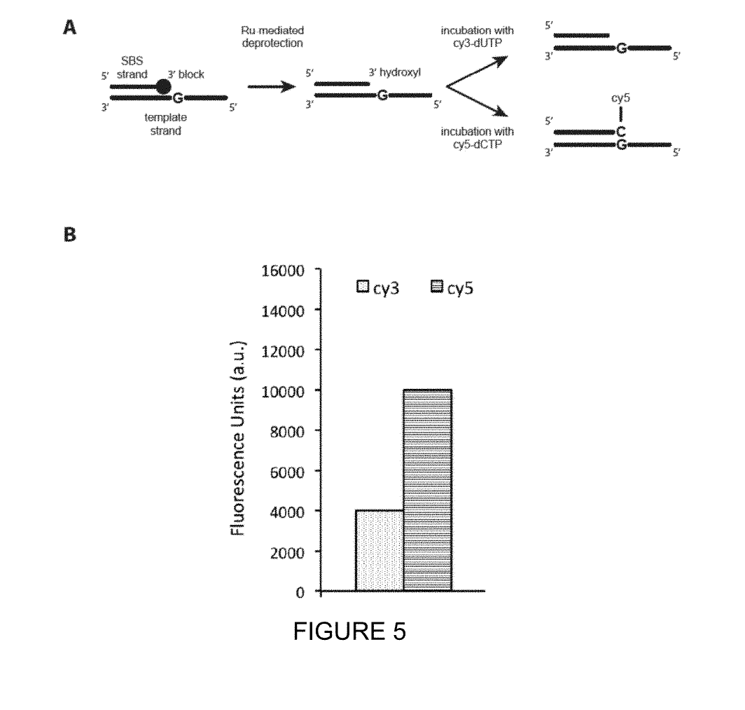

A duplex mimicking an intermediate in the sequencing-by-synthesis (SBS) process, whereby a template strand is immobilised on a solid support and a sequencing primer with a reversibly blocked 3′ terminus is annealed, was incubated with a solution containing a Ru²⁺ species, sodium ascorbate and tris-HCl. The reaction mixture was illuminated at 455 nm for 5 minutes to promote the photoactivation of the ruthenium species that results in cleavage of the reversible terminating group. The identity of the following nucleotide in the template strand was then interrogated by incubation with either complementary or non-complementary fluorescent nucleotides, and a polymerase. Incorporation of fluorescence provided a readout of the sequence (FIG. 5).

The ruthenium-mediated unveiling of a hydroxyl from an azidomethyl terminating group enabled the identification of the following guanine nucleotide in the template DNA strand via the differential incorporation of fluorescently labelled cy3-dUTP and cy5-dCTP. Thus, ruthenium-mediated azidomethyl deprotection finds great utility in sequencing by synthesis processes.

1. A method of converting an azidomethyl ether substituent to a free hydroxyl group wherein said method comprises the step of exposing a compound having said azidomethyl ether substituent to a photoactivated transition metal complex.

2. The method as defined in claim 1, wherein the photoactivated transition metal complex comprises a transition metal selected from ruthenium, platinum, palladium, rhodium and osmium.

3. The method as defined in claim 2, wherein the transition metal is ruthenium.

4. The method as defined in any one of claims 1 to 3, wherein the photoactivated transition metal complex comprises a ligand which is a mono-dentate or bidentate ligand selected from phosphine, thiocynate, nitrogen, pyridine, phenanthroline, cyclopentadienyl and N-heterocyclic carbine based ligands.

5. The method as defined in claim 4, wherein the photoactivated transition metal complex comprises a pyridine ligand, such as a bipyridine ligand.

6. The method as defined in claim 5, wherein the photoactivated transition metal complex is tris(2,2′-bipyridyl)ruthenium(II)).

7. The method as defined in any one of claims 1 to 6, wherein the azidomethyl ether is present on a ribose or deoxyribose sugar moiety.

8. The method as defined in claim 7, wherein the azidomethyl ether is a 2′ or 3′-O-azidomethyl.

9. The method as defined in any one of claims 1 to 8, which comprises the step of exposing a compound having said azidomethyl ether substituent to a photoactivatable transition metal complex followed by photoactivating said photoactivatable transition metal complex.

10. The method as defined in claim 9, wherein said photoactivating is performed with electromagnetic radiation, such as UV and visible light between 400 and 500 nm, such as 450 to 500 nm, in particular 450 nm.

11. The method as defined in claim 10, wherein said photoactivating is performed at 452 nm±5 nm.

12. The method as defined in any one of claims 9 to 11, wherein said photoactivating is performed at a temperature of between 5 and 95° C., such as 20 to 60° C., in particular 22° C.

13. The method as defined in any one of claims 9 to 12, wherein said photoactivating is controlled by a digital micromirror device, a photolithographic mask, a light emitting diode (LED), an LED array, a laser, or a laser array.

14. The method as defined in any one of claims 1 to 13, wherein said transition metal complex is used in combination with a suitable electron donor.

15. The method as defined in claim 14, wherein the suitable electron donor is sodium ascorbate.

16. The method as defined in any one of claims 1 to 15, for use in nucleic acid synthesis.

17. A method of nucleic acid synthesis, which comprises the steps of:
(a) providing an initiator sequence;
(b) adding a nucleotide triphosphate having a 3′-O-azidomethyl substituent to said initiator sequence in the presence of terminal deoxynucleotidyl transferase (TdT) or a functional equivalent or fragment thereof;
(c) removal of TdT;
(d) adding a cleavage composition comprising a photoactivatable transition metal complex and a suitable electron donor;
(e) cleaving the 3′-O-azidomethyl substituent by photoactivating said photoactivatable transition metal complex; and
(f) removing the cleavage composition.

18. The method as defined in claim 17, wherein greater than 1 nucleotide is added by repeating steps (b) to (f).

19. The method as defined in any one of claims 1 to 18, which is performed in a microfluidic device.

20. A method of nucleic acid synthesis which is performed in a microfluidic device comprising the steps of:
(a) providing an initiator sequence bound to a surface within a microfluidic device;
(b) adding a nucleotide triphosphate having a 3′-O-azidomethyl substituent to said initiator sequence in the presence of terminal deoxynucleotidyl transferase (TdT) or a functional equivalent or fragment thereof;
(c) removal of TdT;
(d) adding a cleavage composition comprising a photoactivatable transition metal complex and a suitable electron donor;
(e) cleaving the 3′-O-azidomethyl substituent from a sub-set of nucleotide triphosphates having a 3′-O-azidomethyl substituent by selective photoactivation of a sub-set of said photoactivatable transition metal complexes; and
(f) removing the cleavage composition.

21. The method as defined in claim 19 or claim 20 wherein said microfluidic device is selected from a continuous-flow microfluidic device, droplet-based microfluidic device, digital microfluidic device, microarray device (such as a DNA chip), optofluidic device and acoustic droplet ejection (ADE) device.

22. A kit comprising a nucleotide triphosphate having a 3′-O-azidomethyl substituent and a photoactivatable transition metal complex as defined in any one of claims 1 to 6, optionally in combination with one or more components selected from: an initiator sequence, TdT, a suitable electron donor and a microfluidic device or chip; further optionally together with instructions for use of the kit in accordance with the method as defined in claims 16 to 21.

23. Use of a kit in a method of nucleic acid synthesis, wherein said kit comprises a photoactivatable transition metal complex as defined in any one of claims 1 to 6, optionally in combination with one or more components selected from: an initiator sequence, one or more nucleotide triphosphates having a 3′-O-azidomethyl substituent, TdT, a suitable electron donor and a microfluidic device or chip; further optionally together with instructions for use of the kit in accordance with the method as defined in any one of claims 18 to 23.

24. The method as defined in any one of claims 1 to 15, for use in nucleic acid sequencing.

25. The method as defined in claim 24, wherein the azidomethyl ether is present on the ribose or deoxyribose sugar moiety of a nucleotide or nucleoside.

26. The method as defined in claim 24 or claim 25, wherein the azidomethyl ether is present on the ribose or deoxyribose sugar moiety of a nucleotide or nucleoside and said azidomethyl ether is connected to a detectable tag by an azide-containing linker moiety.

27. The method as defined in any one of claims 24 to 26, wherein treatment with a reducing agent, such as a photoactivated transition metal complex in the presence of a suitable electron donor, simultaneously exposes a 3′ hydroxyl group and decouples the nucleotide or nucleoside from the detectable tag.

28. A method of cleaving an azide-containing linker moiety wherein said method comprises the step of exposing a compound having said azide-containing linker moiety to a photoactivated transition metal complex and suitable electron donor.

29. The method as defined in claim 28, wherein said azide-containing linker moiety is R¹—X—CHN₃—R², wherein X is O, NH, NR³ or S, R¹ is a nucleotide or nucleoside, R² is a detectable label, for example a fluorophore and R³ is an optionally substituted C₁₋₁₀ alkyl group.

30. Use of a kit in a method of nucleic acid sequencing, wherein said kit comprises a photoactivatable transition metal complex as defined in any one of claims 1 to 6, optionally in combination with one or more components selected from: an initiator sequence, one or more nucleotide triphosphates having a 3′-O-azidomethyl substituent, a suitable electron donor and a microfluidic device or chip; further optionally together with instructions for use of the kit in accordance with any of the methods defined in any one of claims 18 to 23.
